(19) United States
(12) Patent Application Publication
(10) Pub. No.: US 2024/0400894 A1
(43) Pub. Date: Dec. 5, 2024

(54) FLUORESCENT MATERIAL FOR RATIOMETRIC AND GASEOUS PHASE SENSING OF VOC

(71) Applicant: COUNCIL OF SCIENTIFIC AND INDUSTRIAL RESEARCH, New Delhi (IN)

(72) Inventors: Madhulekha Gogoi, Jorhat (IN); Lakshi Saikia, Jorhat (IN); Garikapati Narahari Sastry, Jorhat (IN)

(21) Appl. No.: 18/731,015

(22) Filed: May 31, 2024

(30) Foreign Application Priority Data
Jun. 2, 2023 (IN) 202311038420

(51) Int. Cl. C09K 11/06; G01N 21/33; G01N 33/00

(52) U.S. Cl. CPC C09K 11/06; G01N 21/33; G01N 33/0047; C09K 2211/1007

(57) ABSTRACT

The specification discloses a fluorescent material having a europium precursor and a structure directing ligand comprising functionalized graphitic carbon nitride (g-C₃N₄). The specification also discloses a process for making the fluorescent material. Further, the specification discloses a process for using the fluorescent material to sense volatile organic compounds (VOC) in a sample. The process includes preparing the fluorescent material and sensing the VOC in the sample using the fluorescent material. The VOC is 1,4-dioxane.

This application claims priority from the Indian patent application No. 202311038420 dated Jun. 2, 2023.

### TECHNICAL FIELD

The present disclosure relates to a composite for making an Eu-based metal organic framework (MIL-LIC MOF). The present disclosure also relates to a process of synthesizing the composite comprising of g-C₃N₄ and MoS₂ along with europium ions and terephthalic acid, useful for ratio metric sensing and gaseous phase detection of 1,4-dioxane.

### BACKGROUND

Metal organic frameworks (MOF) have found wide applications in molecular sensing (H. Y. Li, S. N. Zhou, S. Q. Zang and J. Li, Chem. Soc. Rev. 49, 6364, 2020), gas storage/separation (J. Li, P. M. Bhatt, J. Li, M. Eddaoudi and Y. Liu, Adv. Mater. 32, 2002563, 2020) and photocatalysis (E Whelan, F. W. Steuber, T. Gunnlaugsson and W. Schmitt, Coordination Chem. Rev. 437, 213757, 2021). However, few drawbacks are still there for pure MOFs such as low photoresponsivity, high recombination rate of electron-hole pair and poor stabilities. Therefore, efforts have been made to develop composites of MOF with semiconductor compounds to form hybrid materials (J. Cai et al. New J. Chem. 41, 3882, 2017) (M. Jahan et al. J Am. Chem. Soc. 132, 14487, 2010) for better performance efficiency.

Reference is made to G. Zhou et al. (App. Cat. B 220, 607, 2018) wherein a novel composite catalyst was developed by combining the MOFs of NH₂-MIL-125(Ti) with g-C₃N₄ functionalized by benzoic acid. Hence, the composite was formed through covalent bonds for the first time, where benzoic acid functionalized g-C₃N₄ acts as the structure directing agent in MOF formation as well. Their results show that with increasing amount of g-C₃N₄, the surface of NH₂-MIL-125(Ti) is gradually covered with nanosheets indicating a good connectivity between the two and also 6 times higher H₂ productivity by the composite than the pure MOF.

Another reference is made to Z. Gao et al. (App. Cat. B 268, 118462, 2020) wherein a composite was prepared by combining the aminated flower-like ZnIn₂S₄ with g-C₃N₄ functionalized by benzoic acid through amide bonds. The composite possesses improved photocatalytic activity due to intimate interfacial contact and well-matched band-gap structure between the two motifs.

Yet another reference is made to X. Liao et al. (Appl. Surface Sci. 503, 144089, 2020) wherein shape-controlled Fe-based MOF was fabricated by the assist of benzoic acid functionalized g-C₃N₄ where the structure directing property of the latter was envisaged. During this work, the authors developed diamond like structure of the composite, (101) surface oriented and exhibiting high photocatalytic activity under visible light irradiation.

None of the above work has tried the fabrication of lanthanide-based MOF with composition of functionalized g-C₃N₄. Another disadvantage of the above-mentioned products is that the functionalized g-C₃N₄ is playing the role of co-structure directing agent along with the primary ligand. It has not been examined whether benzoic acid functionalized g-C₃N₄ alone can participate in the MOF formation.

In addition to this, lanthanide-based MOFs are generously used for sensing applications. 1,4-dioxane is an industrial solvent for inks, adhesives, fats, waxes etc. and is an environmental water toxin due to its high solubility at low vapor pressure and hydrophilicity. It has been identified as a probable human carcinogen by IARC (International Agency for Research on Cancer). Therefore, it is very useful to detect it by a suitable scientific method.

References are made to M. R. Karim et al. (Talanta, 193, 64, 2019) wherein PAni-SiO₂ nanocomposite was deposited onto glassy carbon electrode and the sensor analytical performance was carried out in the presence of different concentrations of 1,4-dioxane.

Another reference was made to a research group from King Abdulaziz University, Saudi Arabia, who have developed various systems for electrochemical sensing of 1,4-dioxane selectively; namely, NiO@Nd₂O₃ nanocomposite (M. M. Rahman et al. New J. Chem. 43, 17395, 2019), mixed metal oxide of ZnO/NiO/MnO₂ nanoparticle (M. M. Rahman et al. RSC Adv. 9, 42050, 2019) and doped ZnO/GO nanocomposite (M. M. Alam et al. J. Mater Sci: Mater Electron 33, 4360, 2022).

Yet another reference is made to D. Ma et al. (ACS Appl. Mater. Interfaces 8, 24097, 2016) who synthesized a porous organic polymer and to S. Shanmugaraju et al. (Chem. Comm. 55, 12140, 2019) wherein Tröger's base functionalized triazine covalent organic polymer was synthesized and employed as a ‘turn-on’ fluorescent sensor of 1,4-dioxane. M. Yun et al. (Appl. Surface Sci. 537, 147872, 2021) is also referred for similar ‘turn-on’ fluorescence sensing of the same using carbon nano-onion. A very recent reference is made to U. Mondal et al. (Dalton Transaction 51, 2083, 2022) where d¹⁰-MOF was used in the same direction.

Main drawback of electrochemical sensing is its applicability in limited temperature range in spite of its reliability and accuracy. The drying out of electrodes induced by severe temperatures and low humidity shortens the lifespan of such a technology. The inherent disadvantages of this process involve tedious job of electrode development and selectivity towards the specific analyte. Moreover, it is necessary to remove the products formed as a result of electrode reactions. On contrary to this, fluorescence-based sensing is simple, quick and cost effective as well. However, simple fluorescence-based sensing via monitoring the intensity change of single fluorescence peak in presence of analyte is highly influenced by external conditions viz. instrument parameter, photo transformation, receptor concentration, competing analytes and autofluorescence as well.

### SUMMARY

In one aspect, the present disclosure provides a metal organic framework (MOF) from lanthanide metal ions and functionalized 2-D sheets of g-C₃N₄.

In another aspect, a process of MOF formation is disclosed, wherein the functionalized g-C₃N₄ acts as the structure directing agent without the use of any other organic molecule.

In yet another aspect, a process for ratiometric sensing of environmental toxin 1,4-dioxane is disclosed.

In yet another aspect, a process is provided wherein the ratiometric sensing with emission peaks of the sensor has the advantage of easy-to-differentiate color and intensity changes.

In another aspect, a detection technique of 1,4-dioxane in gaseous phase using the same compound is disclosed.

In yet another aspect, the mechanism behind the ratiometric sensing and gaseous phase sensing exhibited by the developed MOF compound is disclosed.

In accordance with the purpose(s) of the present disclosure, as embodied and broadly described herein, embodiments of the present invention relate to a lanthanide-based metal organic framework developed from lanthanide metal ions and ligands comprising of functionalized g-C₃N₄, mixture of functionalized g-C₃N₄ with TMDs (transition metal disulfides) and mixture of functionalized g-C₃N₄ with organic molecule. Also, the present invention provides an efficient method of sensing of environmental toxin 1,4-dioxane as an application of the developed MOF and the like.

In some embodiment, the functional group is selected as benzoic acid to be functionalized over g-C₃N₄ which is working as the ligand in MOF formation.

In an embodiment, the ligand comprises of a mixture of functionalized g-C₃N₄ and transition metal disulfide.

In one embodiment, the transition metal disulfide is used as MoS₂.

In another embodiment, the ligand comprises of a mixture of functionalized g-C₃N₄, MoS₂ and an organic molecule.

In yet another embodiment, the organic molecule used is terephthalic acid.

In yet another embodiment, the present disclosure provides a composition comprising Eu-based metal organic framework (MIL-LIC MOF) exhibiting multiple emission peaks useful for sensing applications.

In still another embodiment, the present specification provides a composition comprising Eu-based metal organic framework (MIL-LIC MOF) exhibiting ratiometric sensing of environmental toxin.

In still another embodiment, the present specification provides a composition comprising Eu-based metal organic framework (MIL-LIC MOF) exhibiting gaseous phase detection of volatile environmental toxin.

### BRIEF DESCRIPTION OF THE DRAWINGS

For better understanding of the disclosures and the advantages thereof, the following detailed descriptions with the accompanying drawings have been put forwarded:

FIG. 1A shows the XRD pattern of MZN-1 MOF revealing the presence of mainly g-C₃N₄ phase.

FIG. 1B shows the XRD pattern of MZN-2 MOF which is in good agreement with that of MOF-LIC-1 type.

FIG. 2A shows the SEM image of MZN-1 as a mixture of big and small spheres of sizes ˜1 μm and 250 nm

FIG. 2B shows the TEM image of MZN-1

FIG. 2C shows the SEM image of MZN-2 where the particle size is in the range of 0.5 to 1 μm.

FIG. 2D shows the TEM image of MZN-2 from where the sheets of g-C₃N₄—MoS₂ composites are clearly observed embedding the MOF particles.

FIG. 3A is the comparative XPS plot of C1s of BCN, MZN-1 and MZN-2.

FIG. 3B is the comparative XPS plot of N1s of BCN, MZN-1 and MZN-2.

FIG. 3C is the comparative XPS plot of O1s of BCN, MZN-1 and MZN-2.

FIG. 3D is the comparative XPS plot of Eu3d of MZN-1 and MZN-2.

FIG. 3E is the XPS plot of S2p of MoS₂ present in MZN-2.

FIG. 4A presents the UV-visible absorption spectra of BCN and MSBCN with peaks at 210 nm, 248 nm and 317 nm.

FIG. 4B presents the comparative absorption spectra between BCN and MZN-1.

FIG. 4C presents the comparative absorption spectra between MSBCN and MZN-2.

FIG. 5A shows the comparative fluorescence spectra of BCN and MZN-1, excitation 250 nm.

FIG. 5B shows the comparative fluorescence spectra of MSBCN and MZN-2, excitation 250 nm.

FIG. 5C shows the excitation spectra of MSBCN, MZN-1 and MZN-2.

FIG. 6A shows the FTIR spectra of BCN and MZN-1 from where the benzoic acid functionalization over g-C₃N₄ was observed and the metal carboxylate linkages in MOF formation were confirmed.

FIG. 6B shows the FTIR spectra of MSBCN and MZN-2, which revealed the presence of all MSBCN stretches along with metal-carboxylate bonds in case of MZN-2.

FIG. 7A shows the positive zeta potential value of MZN-1.

FIG. 7B shows the positive zeta potential value of MZN-2.

FIG. 8A illustrates the change in fluorescence spectra of MZN-2 in water upon addition of increasing volume of 1,4-dioxane (10-100 μL), excitation 250 nm.

FIG. 8B illustrates the change in fluorescence spectra of MZN-2 in water upon addition of increasing volume of 1,4-dioxane (2-20 μL), excitation 250 nm.

FIG. 8C shows the response time in changing fluorescence nature of MZN-2 in water upon addition of 1,4-dioxane, excitation 250 nm.

FIG. 9 shows the fluorogenic change of MZN-2 solution in water from pink to blue upon dioxane addition, irradiation with 250 nm.

FIG. 10 shows the selectivity of MZN-2 MOF towards dioxane with the maximum fold change in intensity of both 504 and 617 nm emission peaks in a pool of 16 solvents, excitation 250 nm.

FIG. 11A presents the comparative plot between absorption spectra of dioxane (absorber) and excitation of MZN-2 (fluorophore), demonstrating no inner filter effect on fluorescence quenching mechanism.

FIG. 11B presents the comparative plot between absorption spectra of dioxane (absorber) and emission of MZN-2 (fluorophore), demonstrating no inner filter effect on fluorescence quenching mechanism.

FIG. 11C shows the comparative absorption spectra of MZN-2 (acceptor) and emission spectra of dioxane (donor) with no overlapping, nullifying the role of FRET in fluorescence quenching.

FIG. 12A shows the Stern-Volmer plot with linear characteristics, indicating the quenching may be static or dynamic or mixture of both.

FIG. 12B shows the TRPL plot of MZN-2 with reference to emission 617 nm.

FIG. 12C shows the TRPL plot of MZN-2 after dioxane treatment, with reference to emission 617 nm.

FIG. 13A presents the comparative FTIR plot between MZN-2 and dioxane treated MZN-2 in the lower wavenumber range.

FIG. 13B presents the comparative FTIR plot between MZN-2 and dioxane treated MZN-2 in the higher wavenumber range.

FIG. 14A shows the comparative XPS spectra of C1s between MZN-2 and dioxane treated MZN-2 revealing the presence of O—C—O peak at 285 eV in the latter corresponding to dioxane.

FIG. 14B shows the comparative XPS spectra of N1s between MZN-2 and dioxane treated MZN-2.

FIG. 14C shows the comparative XPS spectra of Eu3d between MZN-2 and dioxane treated MZN-2.

FIG. 14D shows the comparative XPS spectra of O1s between MZN-2 and dioxane treated MZN-2 revealing the presence of loosely bound O species.

FIG. 14E shows the comparative XPS spectra of S2p between MZN-2 and dioxane treated MZN-2.

FIG. 15A presents the fold change in absorbance of MZN-2 at 240 nm on addition of increasing volume of dioxane, exhibiting a behaviour observed in case of complex formation.

FIG. 15B presents the Benesi-Hilderbrand plot, from where the association constant, Kₐ is calculated to be 1.605×10⁻⁴ M⁻¹.

FIG. 16 shows the energy level diagram, from where Type I band arrangement is observed after dioxane addition causing fluorescence quenching.

FIG. 17 shows the valence band XPS spectra of MSBCN and MZN-2, from where the valence band positions was calculated.

FIG. 18A shows the rauc's plot of MZN-2, from where band gap energy was calculated.

FIG. 18B shows the cyclic voltammetry plot of 1,4-dioxane from where the valence and conduction band positions were calculated.

FIG. 19 shows the recyclability plot of MZN-2 MOF as sensing material.

FIG. 20 depicts the outline of the self made vacuum chamber in which the gaseous phase sensing of volatile organic compound (VOC) was carried out.

FIG. 21A shows the I-V characteristic curve of VOC sensing by MZN-2 for methanol.

FIG. 21B shows the I-V characteristic curve of VOC sensing by MZN-2 for dioxane.

FIG. 21C shows the I-V characteristic curve of VOC sensing by MZN-2 for air.

### DETAILED DESCRIPTION

The disclosure provides a metal organic framework (MOF) for sensing of environmental toxin 1,4-dioxane. The composition comprises a novel structure directing ligand obtained from one or more 2D sheets of g-C₃N₄ and MoS₂ and optionally from one or more organic compounds of terephthalic acid, 2-aminoterephthalic acid; useful for sensing of dioxane both in liquid and gaseous state.

In an embodiment, the average particle size of the MOF is in the range of about 50 nm to 200 nm.

In another embodiment, the average particle size of the MOF is in the range of about 0.5 μm to 1.5 μm.

In one more embodiment, the MOF is fluorescent in nature.

In an embodiment, the specification provides a product synthesized by using a solvothermal method.

According to another embodiment, the present disclosure provides a method of synthesizing a MOF comprising-functionalization of the first sheets with benzoic acid and optionally making a composite of it with a second sheet material, the n mixing it with an inorganic compound and optionally with an organic compound in a solvent system. The reaction mixture is put under definite temperature condition for definite time. The MOF particles obtained using the method of the present invention are further collected and purified.

According to another embodiment, the present disclosure provides a method of synthesizing MOF using a solvothermal method comprising mixing of functionalized g-C₃N₄ or composite of it with MoS₂ and a europium precursor in solution, adding an organic ligand optionally.

According to yet another embodiment of the present invention, the solvent used in the method comprises of N,N-dimethylformamide.

In an embodiment the method further comprises collecting the MOF particles obtained using the method of the present invention.

In an embodiment, the mixing is carried out by stirring, shaking using magnetic stirrers, vortex mixers, static mixers, sonicator, kitchen mixers, blenders, nano- and microfluidics devices, pouch mixing, any mixer which creates a turbulent flow and the like, and/or a combination thereof.

In an embodiment, the reaction mixture for the synthesis of the MOF is heated at a temperature of 130° C.

In an embodiment, the temperature increment of the reaction mixture from room temperature to 130° C. takes place at the rate of about 1.0 degree/minute to 2.0 degree/minute.

In an embodiment, the method of synthesizing the MOF particles comprises heating of the reaction mixture for about 12 hour to 48 hours.

In an embodiment, the method of synthesizing the MOF particles comprises cooling of the reaction mixture from 130° C. back to room temperature at a rate of 0.15 degree/minute to 2.0 degree/minute.

In an embodiment, the MOF particles obtained using the method of the present invention is optionally separated from the reactants by centrifugation, decantation, filtration, ultrafiltration, dialysis, diafiltration, rotary evaporation, lyophilization, membrane filtration, syringe filtration and the like, and/or a combination thereof.

In an embodiment, the method of purifying the MOF particles is carried out by using at least one solvent.

In an embodiment, the solvent for purifying the MOF particles obtained using the method of the present invention is selected from methanol, ethanol, propanol, isopropanol, acetone, acetonitrile, dimethyl sulfoxide, tetrahydrofuran and the like, and/or a combination thereof.

In an embodiment, the europium precursor employed for the synthesis of the MOF particles is chosen from europium (III) chloride hexahydrate, europium (III) chloride, europium (III) acetate, europium (III) carbonate, europium (III) nitrate, europium (III) oxalate, europium (III) carbonate, europium (III) oxide and/or a combination thereof.

In an embodiment, the weight ratio between the g-C₃N₄ to europium precursor is used in the range of about 1% (w/w) to 10% (w/w) preferably 7% (w/w).

In an embodiment, the composite of g-C₃N₄ and MoS₂ is formed in the range of about 1% (w/w) to 7% (w/w) preferably 7% (w/w).

In an exemplary embodiment, the weight ratio between the composite of g-C₃N₄ and MoS₂ to europium precursor is used in the range of about 1% (w/w) to 7% (w/w) preferably 5% (w/w).

In yet another embodiment, the mole ratio between the europium precursor and the optionally added organic linker terephthalic acid is used as 3:1.

In yet another embodiment, the mole ratio between the europium precursor and the optionally added organic linker 2-aminoterephthalic acid is used as 3:1.

In an embodiment, the MOF particles are obtained by separating the particles from the reactants.

However, the use of other known aqueous solvents compatible for use with the developed MOF particles of the present invention is completely within the scope of the present invention.

According to a further embodiment, the MOF particles exhibit fluorescence in the blue region while being excited by UV light.

In another embodiment, the MOF particles exhibit fluorescence with dual emission peaks in the cyan and orange-red region while exciting by UV light resulting in pink color solution under UV light.

In yet another embodiment, the quantum yield of the developed MOF were found to be 4.1 and 4.8%.

According to another embodiment, the developed MOFs help in sensing of 1,4-dioxane i.e. the composition is capable of or has the ability to be used in detection of environmental toxin and carcinogenic solvent 1,4-dioxane.

According to an embodiment, the sensing action is performed by the ratiometric change in intensity of emission peaks of the MOF solution in presence of the analyte 1,4-dioxane.

In an embodiment, the sensing action is realized by colorimetric change of the solution from pink to blue in presence of 1,4-dioxane under UV light.

In yet another embodiment, the mechanism behind the ratiometric fluorescence sensing consists of static quenching of metal emission caused by complex formation between MOF and analyte 1,4-dioxane; whereas fluorescence enhancement caused by superimposition of the ligand emission peak with that of the emissive analyte 1,4-dioxane.

In an exemplary embodiment, the MOF particles comprising of composite of g-C₃N₄ and MoS₂ along with europium ions and terephthalic acid exhibit excellent selectivity for the concerned analyte 1,4-dioxane in the solvent pool of water, ethanol, methanol, acetonitrile, ethyl acetate, N,N-dimethylformamide, propanol, isopropanol, butanol, dichloromethane and/or a combination thereof.

In an embodiment, the LOD of the sensing experiment was calculated to be 0.3 μM.

In an embodiment, the MOF particles comprising g-C₃N₄ and europium ions can be used for sensing of gaseous phase 1,4-dioxane as it is a volatile liquid.

In another embodiment, the MOF particles comprising composite of g-C₃N₄ and MoS₂ along with europium ions and terephthalic acid can be used for sensing of gaseous phase 1,4-dioxane as it is a volatile liquid.

According to a further embodiment, the MOF particles exhibit gaseous sensing by changes in current vs. voltage (I-V) characteristics in air and in the presence of the volatile organic compound 1,4-dioxane.

In an embodiment, the MOF particles comprising of composite of g-C₃N₄ and MoS₂ along with europium ions and terephthalic is used for simultaneous detection of 1,4-dioxane in liquid and gaseous phase i.e. the composition is able to or capable of being used for simultaneous detection of 1,4-dioxane in liquid and gaseous phases.

The technical features of the specification are:
1. g-C₃N₄ functionalized with benzoic acid can serve as the sole structure directing ligand in MOF formation eliminating the need of any organic linker molecule.
2. The composition comprising of composite of g-C₃N₄ and MoS₂ along with europium ions and terephthalic acid exhibits simultaneous detection capability of 1,4-dioxane both in liquid and gaseous phase.
3. The aforementioned detection of 1,4-dioxane in liquid phase has been achieved by ratiometric fluorescence sensing for the first time.

### EXAMPLES

The following examples are given by way of illustration of the present invention and therefore should not be construed to limit the scope of the present invention.

### Example 1: Method of Preparation of Metal Organic Framework

### Example 1A: Synthesis of g-C₃N₄

20 gm (0.26 mol) of thiourea was taken in a silica crucible and heated in a furnace at temperature 550° C. for 2 h with lid covered. After cooling to room temperature, 1.6 gm of yellow powder of bulk C₃N₄ was obtained. This was again heated at 550° C. for 2 h with lid open. Finally, 0.38 gm of white powder of g-C₃N₄ was obtained.

For exfoliation, 129 mg of g-C₃N₄ was dispersed in a mixture of 10 mL water and 10 mL isopropyl alcohol in a 150 ml beaker. Then the solution was ultrasonicated with the help of a probe sonicator in an ice-bath for 48 h (continuous for 30 min, break for 5 min). After sonication, the solution was allowed to stand for 12 h and the precipitate was discarded by decantation. The supernatant was centrifuged at 10,000 rcf for 6 min. The precipitate obtained such was oven dried at 60° C. for 12 h. The powder obtained weighed ˜103 mg.

### Example 1B: Functionalization of g-C₃N₄ with Benzoic Acid (BCN)

1.267 gm (7 mmol) of 2-aminoterephthalic acid and 280 mg of NaOH (7 mmol) were added to 80 mL of water and stirred in ice-bath. When temperature reached 5° C., 526 mg (4 mmol) of NaNO₂ was added to it slowly. Then 6 mL (19.2 mmol, 6.4 M) of 20% HCl solution was added quickly to it followed by 45 min stirring. At the instant of HCl addition, the solution became pale yellow in color and later creamish precipitate was formed. The powder of phenyl carboxylic diazonium salt was obtained by filtration with Whatman 41 (diameter 110 mm) filter paper and oven drying at 60° C. for 12 h.

For functionalizing the carboxylic acid groups on g-C₃N₄ sheets, 300 mg of it was dispersed in 50 mL of 1% (w/v) sodium dodecyl solution in water by sonication. When temperature reached ˜7° C., 300 mg of above prepared phenyl carboxylic diazonium salt was added to it and stirred at 5° C. for 4 h and then at room temperature for another 4 h. The powder of functionalized g-C₃N₄, named it as BCN, was obtained by centrifuging the above solution at 10,000 rcf for 6 min and by oven drying the precipitate at 60° C. for 12 h. Yield is 570 mg.

### Example 1C: Synthesis of Composite of BCN with MoS₂ (MS-BCN)

50 mg BCN and 2.6 mg of MoS₂ were mixed and dispersed in 50 mL ethanol by sonication for 2 h. Then, the solution was heated to evaporate ethanol and then dried in oven for 1 h at 60° C. In this way, 5% (w/w) composite of greyish color was obtained with a yield of ˜40 mg.

### Example 1D: Synthesis of MOF from Eu³⁺ Ions and BCN (MZN-1)

73 mg (1 mmol) of EuCl₃·6H₂O and 7.3 mg (10% by wt) of functionalized g-C₃N₄ (BCN) were added to 50 mL N,N-dimethylformamide and mixed by probe sonication. Then the solution was stirred magnetically for 1 h and then put in an autoclave at 130° C. for 48 h. Creamish precipitate was obtained after centrifugation at 7000 rpm for 10 min and washing with ethanol and acetone. It was oven dried at 60° C. for 12 h. Yield is ˜47 mg.

### Example 1E: Synthesis of MOF from Eu³⁺ Ions, Terephthalic Acid and MSBCN (MZN-2)

264 mg (0.7 mmol) of EuCl₃·6H₂O and 20 mg (7% by wt) of MSBCN were dispersed in 20 mL DMF. It was mixed with a solution of terephthalic acid prepared by dissolving 58 mg (0.35 mmol) of BDC in 40 mL DMF. The resulting solution was stirred at room temperature for one h and then put in autoclave at 130° C. for 48 h. The creamish powder was obtained after centrifugation, washing with ethanol-acetone, oven drying at 60° C. for 12 h. Yield is ˜200 mg.

### Example 2: Characteristics of the MOF Particles

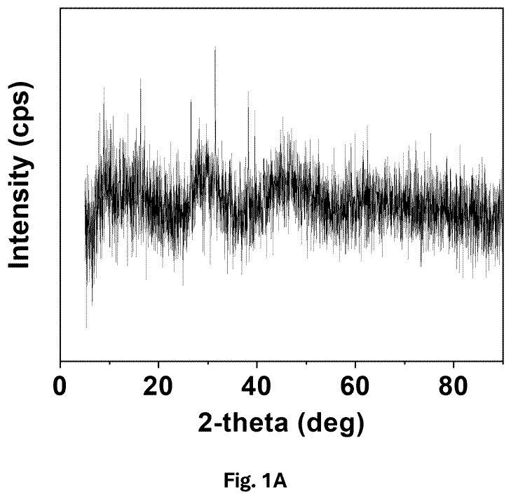

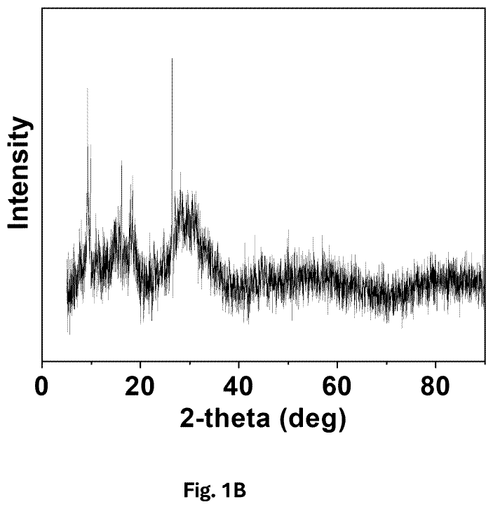

In order to confirm the composition of the MOF X-ray diffraction study was carried out. The X-ray diffraction (XRD) curve as shown in FIG. 1A of MZN-1 reveals the presence of mainly the g-C₃N₄ phase. Whereas the XRD curve of MZN-2 as shown in FIG. 1B is in good agreement with that of MOF-LIC-1 type. However, g-C₃N₄ peaks are also present along with the MOF XRD peaks in case of MZN-2.

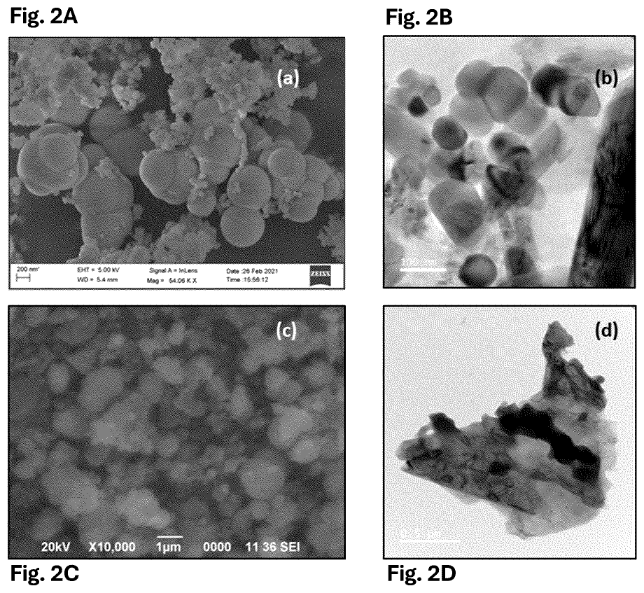

Further, the morphologies of MOFs are characterized by SEM and TEM, as shown in FIG. 2. The results show that MZN-1 is a mixture of big and small spheres of sizes ˜1 μm and 250 nm whereas range of particle size of MZN-2 is 0.5 to 1 μm. From the TEM images of MZN-2 the sheets of g-C₃N₄—MoS₂ composites are clearly observed embedding the MOF particles.

TABLE 1
XPS Peak details of BCN, MZN-1 and MZN-2

| | | | | |
|---|---|---|---|---|
| Sample | Peak | Peak | Peak | Shifting |
| name | position | position | position | from BCN |

C1s

| | | | | |
|---|---|---|---|---|
| BCN | 283.7 eV | 287.1 eV | | |
| | (C—C) | (C—N₃) | | |
| MZN-1 | 284.6 eV | 288.6 eV | 289.9 eV | 0.7 eV, 1.5 eV |
| MZN-2 | 283.7 eV | 287.1 eV | | |

N1s

| | | | | |
|---|---|---|---|---|
| BCN | 397.6 eV | 399.4 eV | 403 e V | |
| MZN-1 | 398.4 eV | 400.2 eV | | 0.8 eV |
| MZN-2 | 397.6 eV | 399.4 eV | | |

O1s

| | | | |
|---|---|---|---|
| BCN | 531.6 eV | 535.5 eV | |
| MZN-1 | 532.6 eV | | 1 eV |
| MZN-2 | 530.7 eV | | 0.9 eV |

Eu3d

| | | |
|---|---|---|
| MZN-1 | 1125.7 eV | 1135.3 eV |
| MZN-2 | 1124.7 eV | 1134.3 eV |

S2p

| | | |
|---|---|---|
| MZN-2 | 161.4 eV | 166.4 eV |

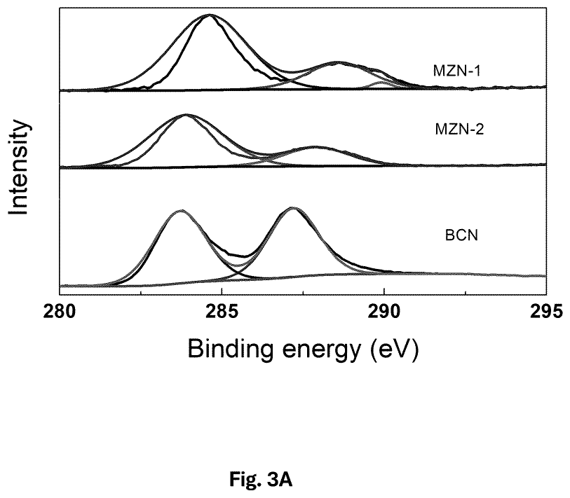

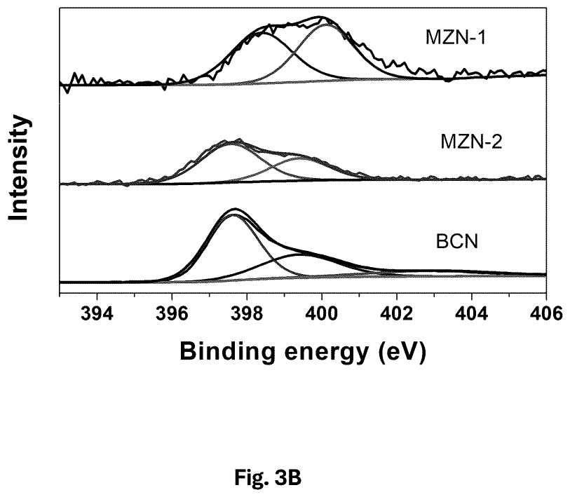

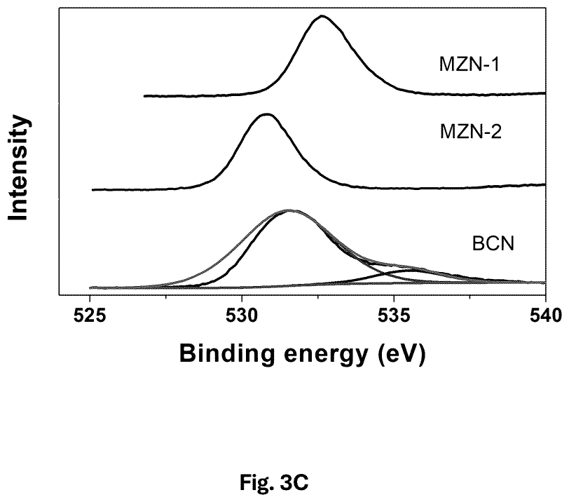

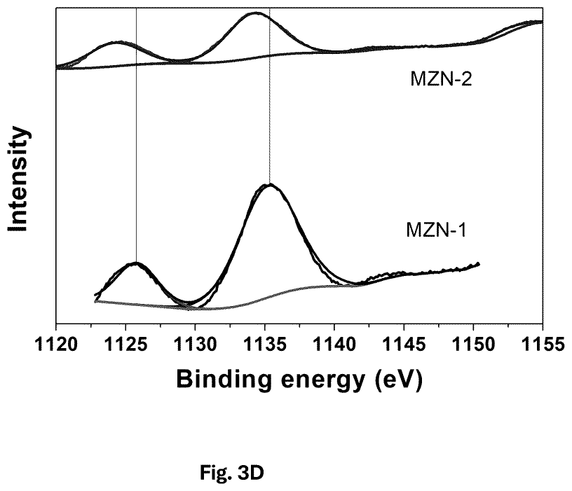

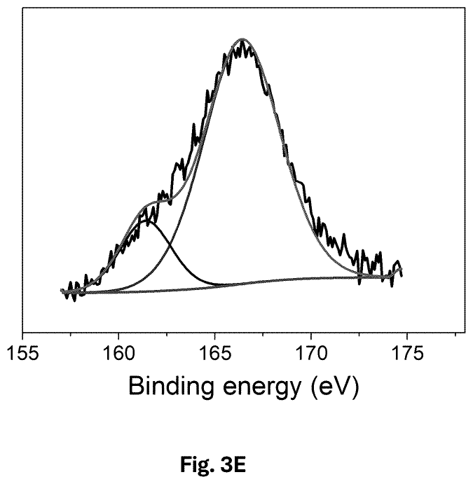

Compositional analysis of the developed MOFs were confirmed from XPS study. FIG. 3 shows the XPS spectra of BCN, MZN-1 and MZN-2 respectively. While the comparative peak positions of the elements in three samples are detailed in Table 1. First of all, presence of O in the XPS spectra of BCN confirms the benzoic acid functionalization over C₃N₄. Also, the C—C position is shifted to lower binding energy from ideal 284 eV indicating C—C defects in C₃N₄. But on MZN-1 MOF formation, this peak is shifted to higher binding energy indicating the coordination between BCN and Eu³⁺ ion. The 287.1 eV peak corresponds to C—N bond which decreases in intensity on MOF formation. On the other hand, the peak for O—C—O linkage is observed only in case of MZN-1 at 289.9 eV. The N1s spectra is presented in FIG. 3B. The C—N═C peak is observed at 397.6 eV and the N—(C)₃ peak at 399.4 eV. Both the peaks are shifted to higher binding energy in case of MZN-1 pointing towards effective coordination between BCN and Eu3+ ion. Whereas the subpeak observed at 403 eV in case of only BCN indicates N—O kind of bonding is also present. From the O1s spectra in FIG. 3C C═O peak is observed at 531.6 eV and a peak at 535 eV for adsorbed oxygen in BCN spectra. On the other hand, O1s spectra is not consistent in both MZN-1 and MZN-2 indicating different types of oxygen linkages in all three samples. Interestingly, C1s and N1s spectra of MZN-2 corroborates with that of BCN which indicates the metal linkage in MZN-2 is through any other atom viz. S—O or such leaving BCN free from metal coordination. However, Eu²⁺3d_{5/2} and Eu³⁺3d_{5/2} peaks at 1125 eV and 1135 eV are present in the Eu3d spectra of both MZN-1 and MZN-2 (FIG. 3D) corroborating to the presence of both Eu²⁺ and Eu³⁺ ion. In the S2p spectra of MZN-2 in FIG. 3E, the peak at 166.4 eV accompanied by a peak at 162 eV corresponds to sulphite ([SO₃]²⁻) and thiosulphite (S—SO³]²⁻). This indicates to an S—O interaction between MoS₂ and BCN in MSBCN. This observation is in support of the assumption that metal coordination with Eu³⁺ is occurring through S—O kind of linkages. Although existence of Mo⁴⁺ peaks are not detected in the survey spectra but elemental compositional analysis reveal the presence with low composition in MZN-2 (Table 2).

TABLE 2
Elemental composition of BCN, MZN-1 and MZN-2 from XPS
BCN

| Elements | C1s | N1s | O1s |
|---|---|---|---|
| Atomic % | 49.6 | 32.1 | 18.3 |

MZN-1

| Elements | C1s | N1s | O1s | Eu3d3 | Eu3d5 |
|---|---|---|---|---|---|
| Atomic % | 51.57 | 1.82 | 45.74 | 0.37 | 0.5 |

MZN-2

| Elements | C1s | N1s | O1s | Eu3d3 | Eu3d5 | S2p1 | S2p | S2p3 | Mo3d3 | Mo3d |
|---|---|---|---|---|---|---|---|---|---|---|
| Atomic % | 43.45 | 2.08 | 41.82 | 0.23 | 0.36 | 6.47 | 2.19 | 3.3 | 0.04 | 0.05 |

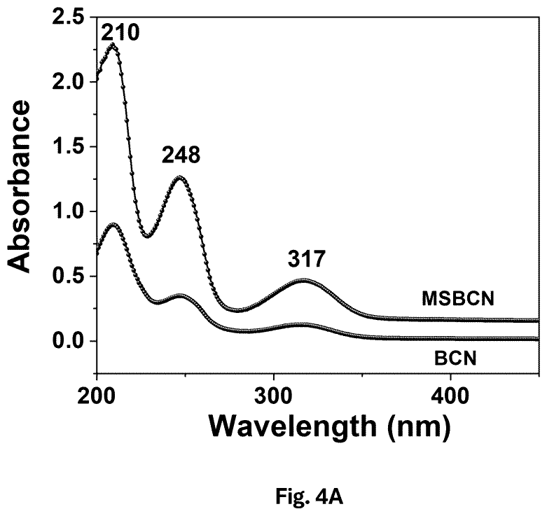

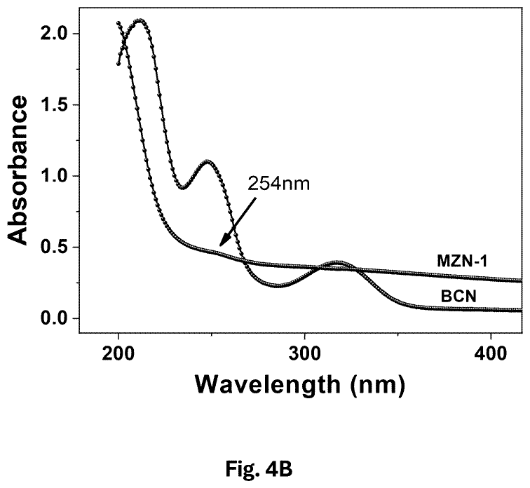

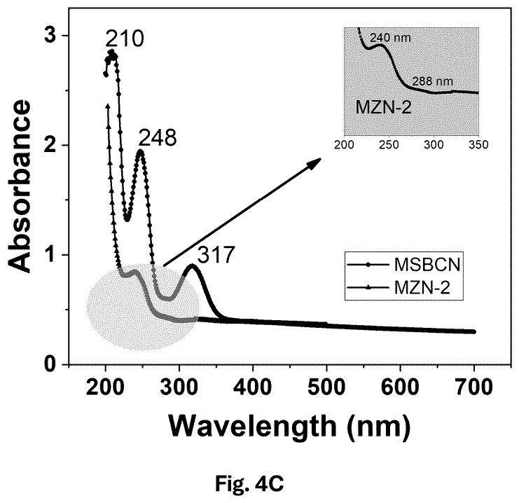

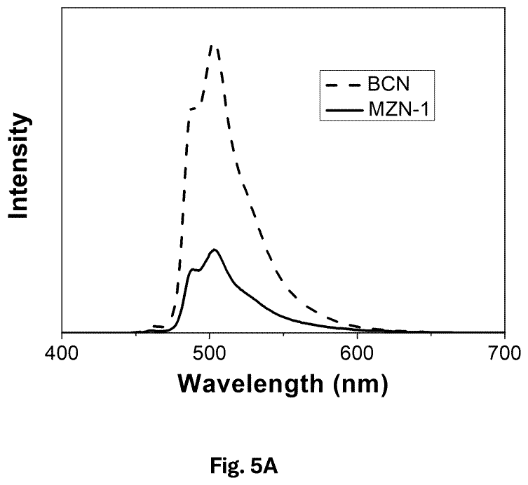

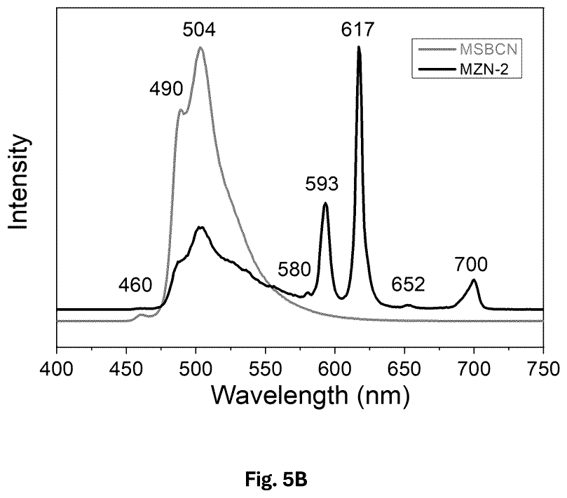

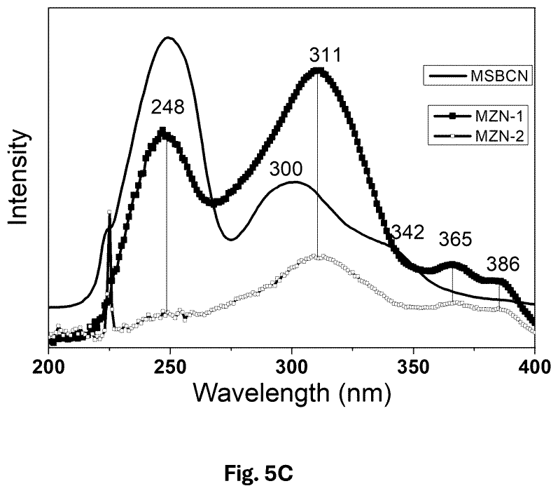

Optical characterization of BCN and MSBCN were done by UV-visible absorption and fluorescence spectroscopy. From the absorption spectra as shown in FIG. 4A, peaks are observed at 210 nm, 248 nm and 317 nm which are due to π to π* and n to π* transitions. While in the absorption spectra of MZN-1 this peak is slightly red shifted to 254 nm exhibiting metal-ligand complex formation whereas the 317 nm peak is intact but decreases in intensity significantly (FIG. 4B). On the other hand, though the ligand MSBCN possesses the same absorption peaks of BCN, but in the absorption spectra of MZN-2, this peak is blue-shifted to 240 nm and the peak at 317 nm disappears, in place of which a new peak appears at 288 nm (inset of FIG. 4C). This confirms the interaction between Eu³⁺ and ligand system in MZN-2. From the fluorescence spectra of BCN and MSBCN as shown in FIGS. 5A and 5B, the emission peaks are observed at 460 nm, 490 nm and 504 nm corresponding to three excitation peaks at 248 nm, 300 nm and 342 nm respectively (FIG. 5C). Whereas the emission spectra of MZN-1 possess only ligand emissions as shown in FIG. 5A but MZN-2 (FIG. 5B) possesses both ligand (504 nm) and metal-centered emissions at 580 nm (⁵D₀ to ⁷F₂), 593 nm (⁵D₀ to ⁷F₁), 617 nm (⁵D₀ to ⁷F₂), 652 nm (⁵D₀ to ⁷F₃) and 700 nm (⁵D₀ to ⁷F₄). This indicates the effective antenna effect in the formation of MZN-2 MOF.

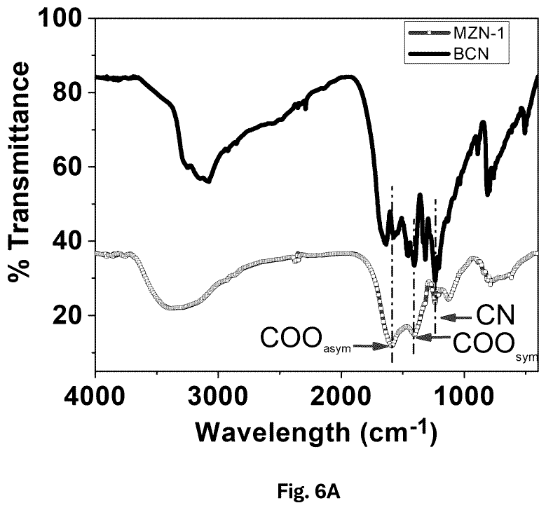

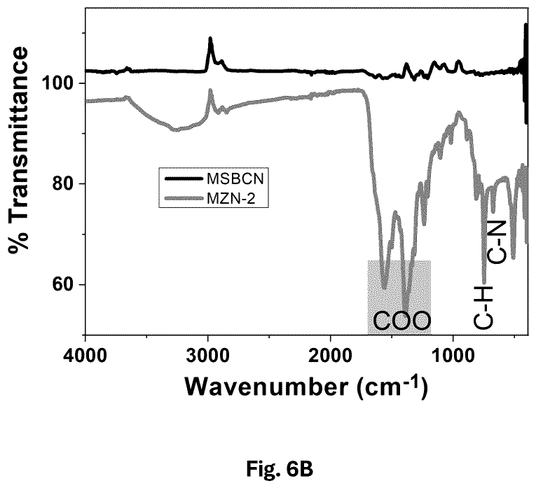

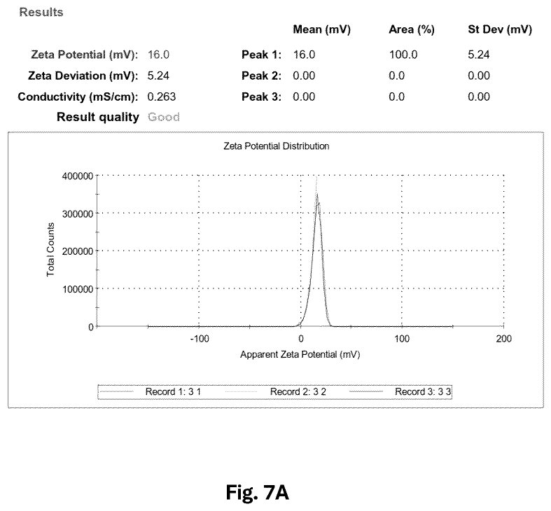

Benzoic acid functionalization over g-C₃N₄ was performed by FTIR spectroscopic analysis. From the FTIR spectra of BCN in FIG. 6A, bending vibration of heptazine unit is observed at 804 cm⁻¹. Additional bands other than C—N of C₃N₄, are observed as doublets at 1453 cm⁻¹, 1463 cm⁻¹, the N═N stretch at 1544 cm⁻¹, the C═C band at 1572 cm⁻¹ and C—O band at 1628 cm⁻¹, which corroborate to the presence of dibenzoic acid group over C₃N₄. Whereas the FTIR spectra of MZN-1 as shown in FIG. 6A, exhibits mainly the presence of COO symmetric and asymmetric stretching bands at 1398 cm⁻¹ and 1578 cm⁻¹ respectively corroborating to the presence of metal carboxylate linkages in the MOF. Also, the C—N stretch at 1203 and 1234 cm⁻¹, C—O stretch at 1125 cm-land bending vibration of heptazine unit at ˜800 cm⁻¹ reveal the formation of MZN-1 with BCN as ligand. In the FTIR spectra of MSBCN as shown in FIG. 6B, all the stretches of BCN are observed with slight shifting in some cases. On the other hand, MZN-2 spectra (FIG. 6B) consists of the peaks of MSBCN along with that of COO symmetric and asymmetric stretching, C—H_{bend} at 742 cm⁻¹, C—N_{stretch} at 670 cm⁻¹ and metal-oxygen stretch at 496 cm⁻¹.

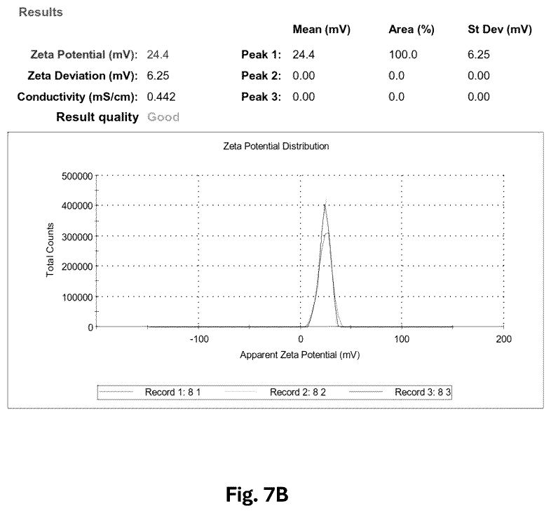

Further surface charge of the developed MOF were studied through zeta potential measurement from where, values were obtained as 16.0 mV and 24.4 mV for MZN-1 and MZN-2 respectively. The results are shown in FIG. 7.

### Example 3: Sensing Activity of Developed MOF

Among the developed MOF of the present invention, MZN-2 was found to be efficient sensor for environmental toxin 1,4-Dioxane both in liquid and vapor phase. In addition to this, MZN-2 exhibited very good gas phase sensing response towards another volatile organic compound methanol as well.

To assess the sensing activity of MOF for liquid phase detection of 1,4-Dioxane, fluorescence spectroscopy was performed on the MOF solution added with analyte. Selectivity study and experiments for LOD calculation were carried out and the results are described below.

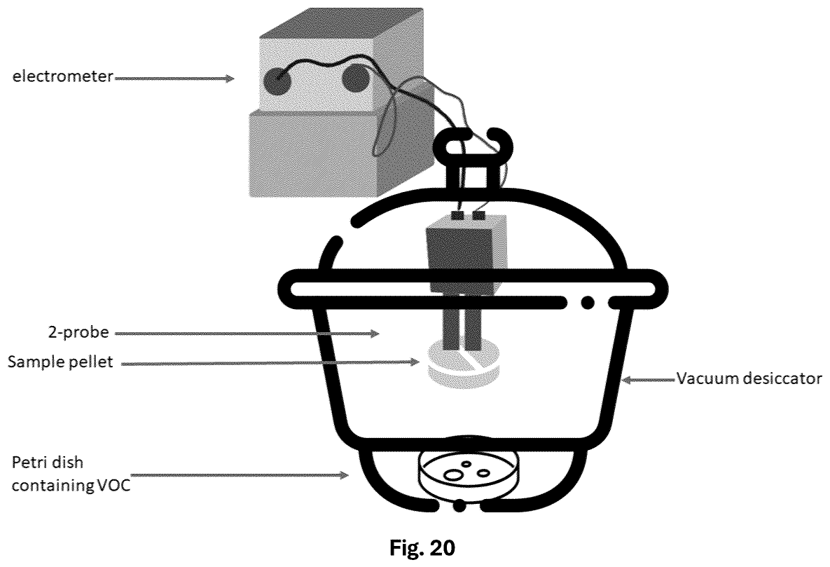

On the other hand, vapor phase detection of volatile liquid like 1,4-Dioxane and methanol was accomplished via I-V characteristics study of the pellets developed from the MOF, by placing it in the respective gaseous environment in a vacuum chamber made by us. The outline of the vacuum chamber is depicted in FIG. 20.

It was observed that the developed MOF MZN-2 exhibited ratiometric sensing behavior in response to 1,4-Dioxane; on introduction of the analyte to the MOF solution, the metal centered emission peaks quenched while the ligand centered emission peak enhanced in intensity. Then the mechanism behind the ratiometric behavior in the emission intensities of the MOF on analyte introduction, was understood by performing a few control experiments and by developing the energy level diagram of the contributing entities.

Moreover, in case of gaseous phase sensing, enhancement of I-V response of the MZN-2 pellet in the environment of volatile organic compound (dioxane and methanol) was seemed to be many fold.

### Example 3A

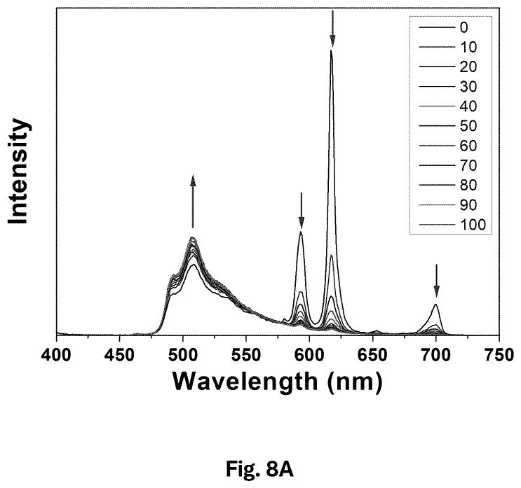

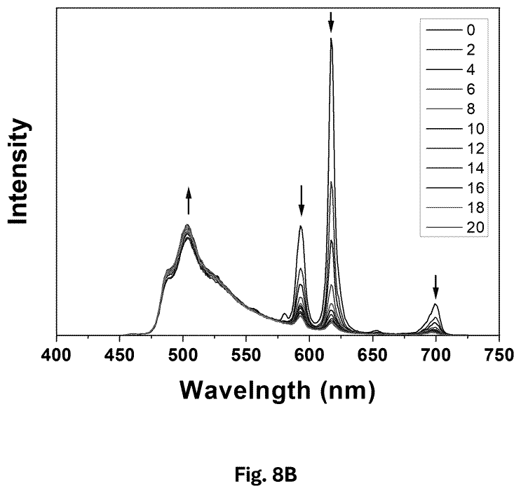

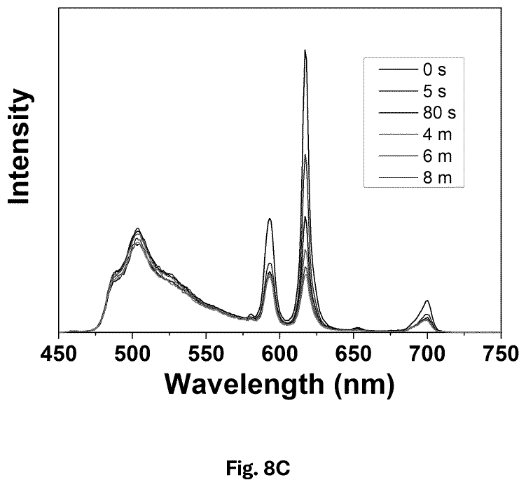

In order to check the fluorescence-based sensing of 1,4-dioxane, a solution of MZN-2 was prepared by dispersing 2.5 mg of it in 2.5 mL water followed by sonication. Then fluorescence spectra was recorded by exciting with 270 nm. The sensing experiment was performed by adding increasing volume of dioxane solution with stirring and recording the fluorescence spectra at same excitation wavelength. The response time was calculated to be as fast as 5 s. The results are shown in FIG. 8.

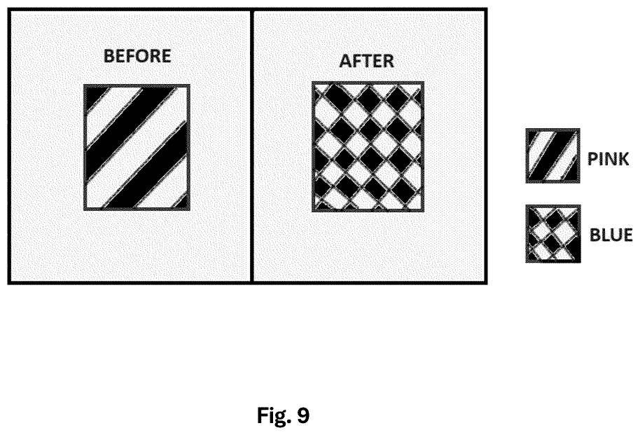

As seen from FIGS. 8A and 8B, on introduction of dioxane to the solution of MOF in water, the metal centered emissions at 593, 617 and 700 nm quenches and on increasing the analyte solution the quenching occurs to zero level. On the other hand, the intensity of the emission peak at 504 nm originating either from metal perturbed ligand emission or due to LMCT increases gradually and saturates after some interval. The response time was seen to be as fast as 5 s from the response time plot of FIG. 8C. This kind of ratiometric sensing is well formulated by taking photographs of the MOF solution in dark condition, excited with UV light. The photographs are shown in FIG. 9, from where it is observed that the solution exhibited purple coloration while after addition of dioxane the exhibited color is blue.

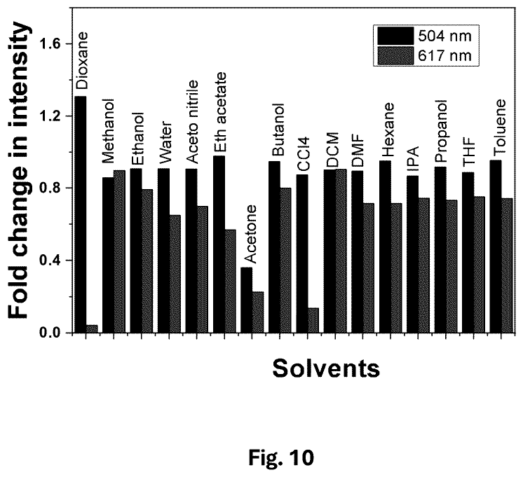

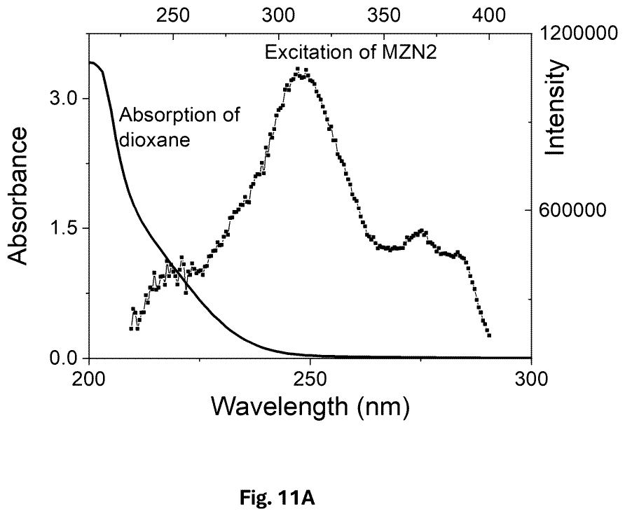

The selectivity results shown in FIG. 10 clearly demonstrate the discriminative sensing ability of the MOF towards dioxane with the maximum fold change in intensity of both 504 and 617 nm emission peaks in a pool of 16 solvents.

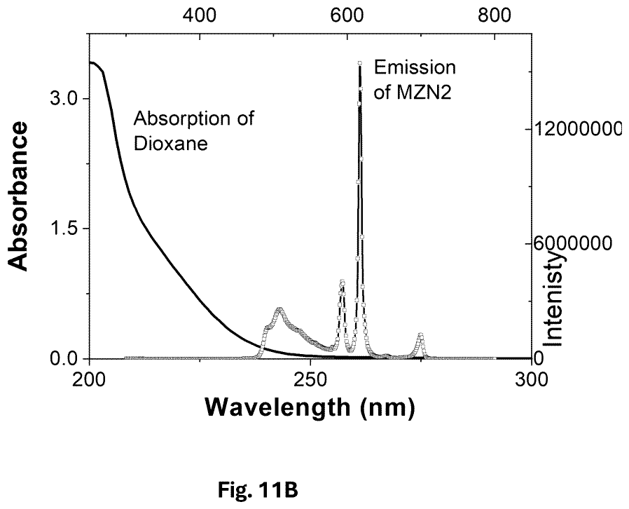

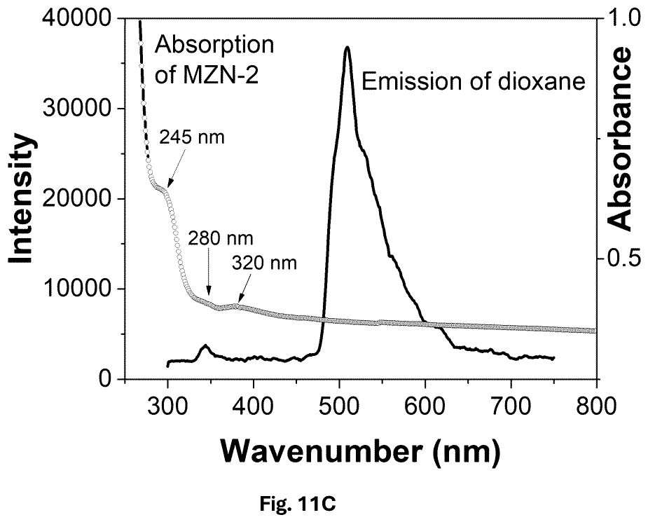

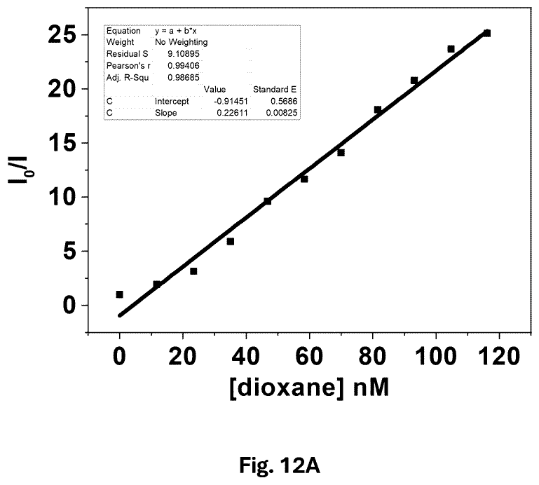

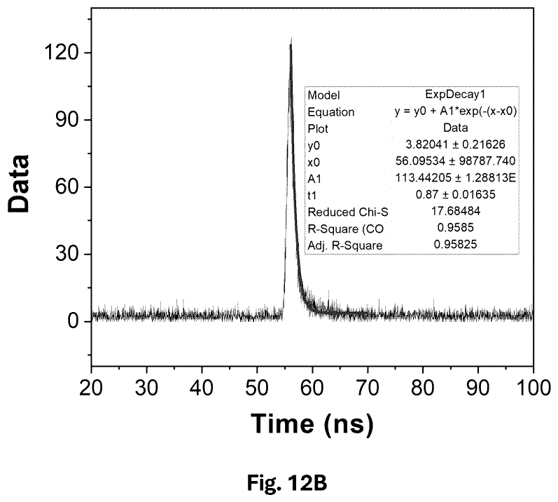

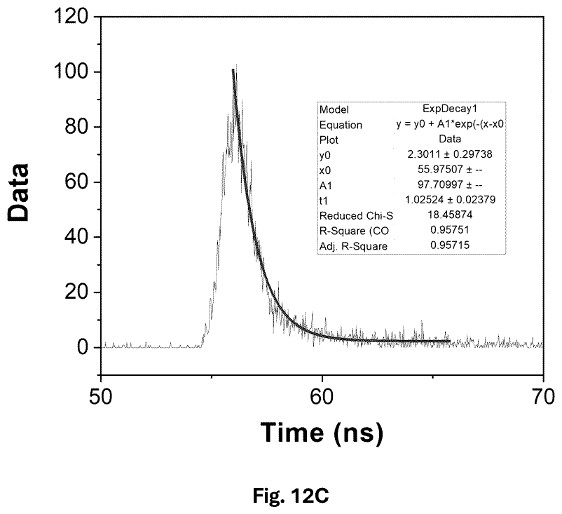

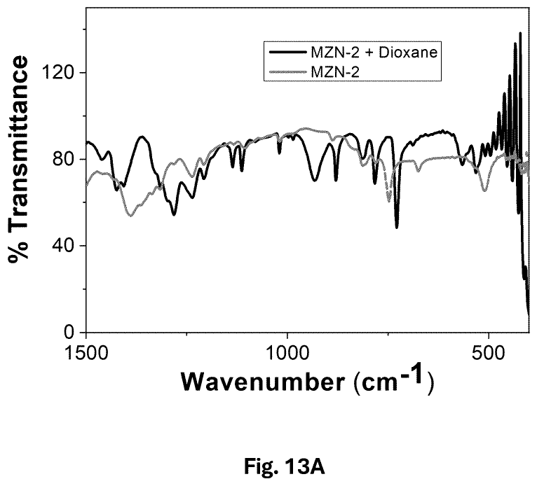

In order to understand the mechanism behind the ratiometric sensing behavior towards 1,4-dioxane, first of all the quenching possibilities through inner filter effect (IFE) and FRET were checked. From the comparative plot between absorption spectra of dioxane (absorber) and excitation (FIG. 11A) and emission (FIG. 11B) of MZN-2 (fluorophore), no overlapping is observed which rules out IFE. Also, FRET possibility is nullified by comparing absorption spectra of MZN-2 (acceptor) and emission spectra of dioxane (donor) with no overlapping at all (FIG. 11C). Then the Stern-Volmer plot is obtained with linear characteristic, shown in FIG. 12A, which indicates the quenching may be static or dynamic or mixture of both. The quenching constant K_{SV} is found to be 2.26×108 M⁻¹. Next, time resolved photoluminescence (TRPL) was carried out with MZN-2 before (FIG. 12B) and after dioxane addition (FIG. 12C) to determine the type of quenching of the metal centered emissions. Specifically, the emission peak at 617 nm was considered and the TRPL plots were fitted with mono exponential decay curves. It was observed that excited state lifetime remained almost constant upon addition of dioxane analyte (0.87 ns to 1.0 ns) indicating the type of quenching as static. Static quenching is caused by complex formation between analyte and fluorophore, forming a non-fluorescent complex. This results into decrease in no of emitting fluorophores and hence causing quenching but not all the fluorophores form complex so ultimately the excited state lifetime of the remaining fluorophores remains almost same.

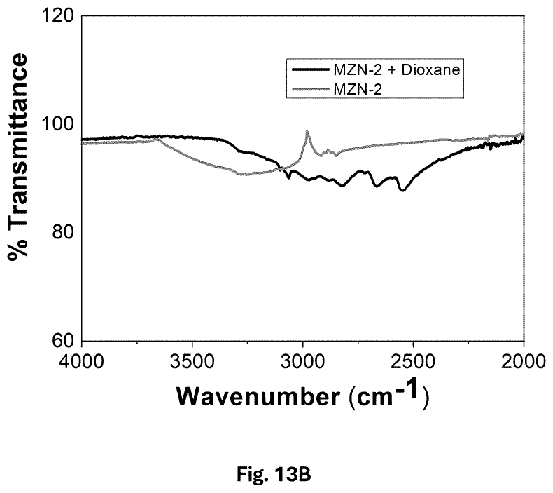

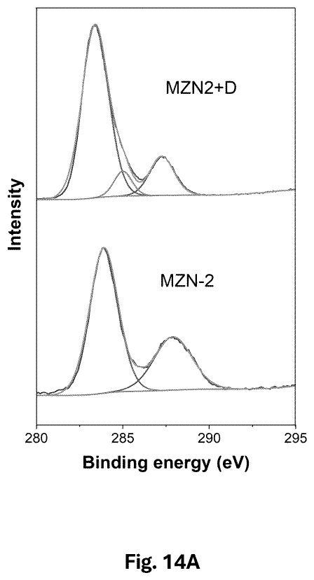

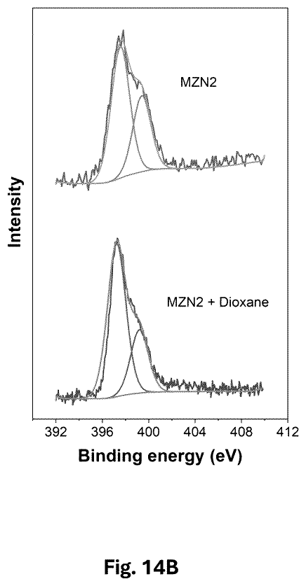

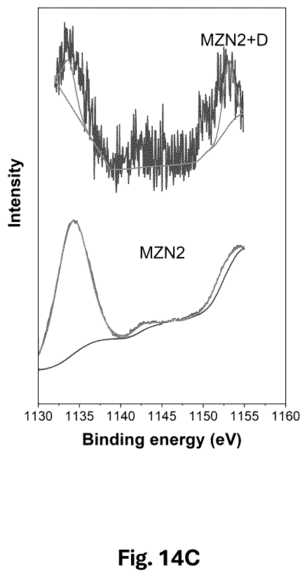

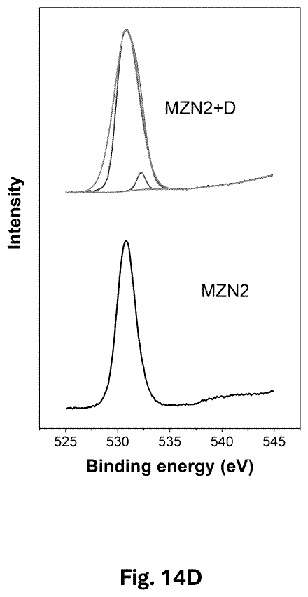

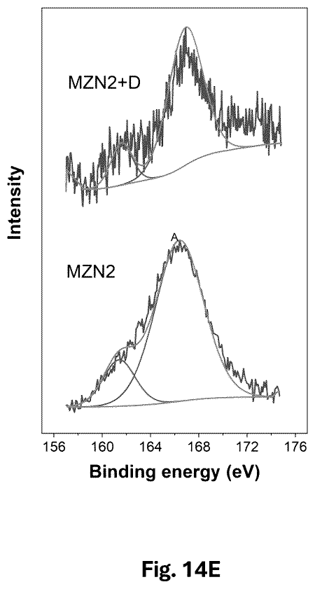

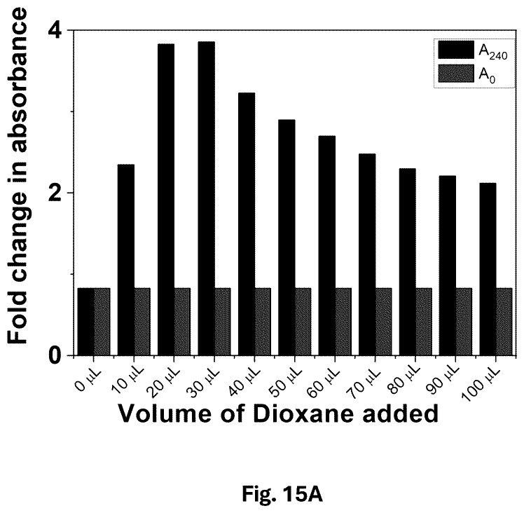

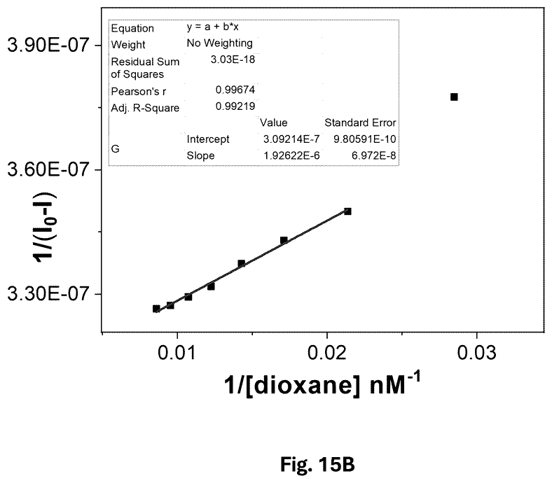

Once the type of quenching appears to be static, next thing is to find out the mechanism for complex formation. In this regard, zeta potential values of MZN-2 and dioxane solution were found to be 24.4 mV (refer FIG. 7B) and −1.61 mV (graph not obtained) respectively. This finding points towards a kind of electrostatic interaction between the two entities. However FTIR spectra of MZN-2 treated with dioxane was recorded and presented in FIG. 13A, from where it is observed that the COO symmetric and antisymmetric stretches at 1372 and 1560 cm⁻¹ disappear which may be due to replacement of coordination bond between carboxylate groups and Eu³⁺ ion in MZN-2 by dioxane coordination. The dioxane complexes of Magnesium recovered from Grignard solution are well established, where both the oxygen atoms of dioxane take part in coordination. Similar kind of interaction is supposed to be happened in this case also corroborated by the appearance of four new absorption peaks in the fingerprint region 400-600 cm⁻¹ for metal-oxygen bonding, in the FTIR spectra of MZN-2 treated with dioxane. Moreover, the presence of C—O stretch for cyclic ether at 933 cm⁻¹ along with the C—O stretches in the region 1100-1200 cm⁻¹ confirms to the presence of dioxane in the treated MZN-2. Also, extended S related peaks are observed in the range of 2500 cm⁻¹ in the FTIR spectra of this sample in comparison with MZN-2, as shown in FIG. 13B. This observation again indicates towards the possibility of interaction between S containing groups of MZN-2 with dioxane. However, from the XPS spectra of MZN-2 and dioxane treated MZN-2, it is observed that C1s spectra (FIG. 14A) of the later possesses a peak at 285 eV corresponding to O—C—O bond revealing the presence of dioxane. Whereas, the O1s spectra (FIG. 14D) of this sample also exhibits an additional peak at 532 eV characteristic of loosely bound oxygen on the surface such as metal-OH groups. However, UV absorption study of MZN-2 with dioxane addition revealed that absorption increases to manifold just after addition of analyte, reaches maximum and then starts decreasing, the results are shown in FIG. 15A. This suggests the possibility of MZN-2 and dioxane complex formation. Also, UV-visible absorption spectra of only MSBCN was recorded by adding increasing volume of 1,4-dioxane which does not exhibit any change at al which confirms the interaction of dioxane is with MZN-2 and not with MSBSN alone. From the Benesi-Hilderbrand plot in FIG. 15B, the association constant, Kₐ is calculated to be 1.605×10⁻⁴ M⁻¹.

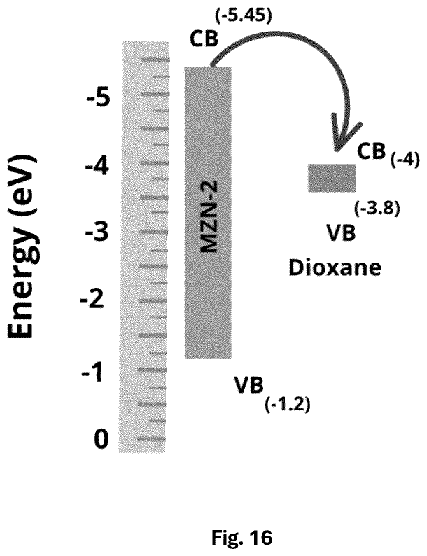

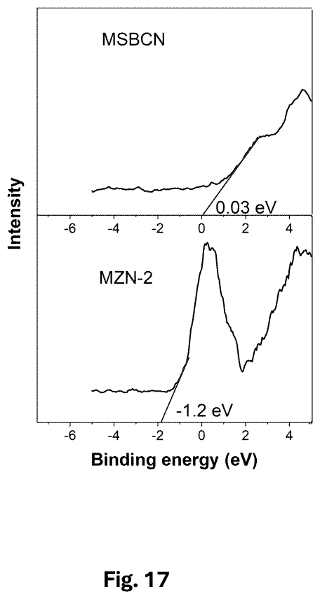

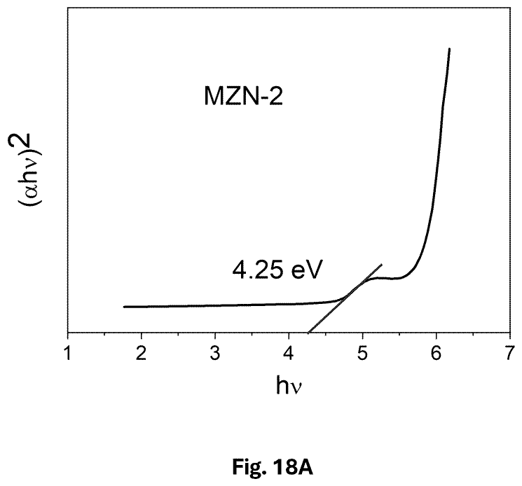

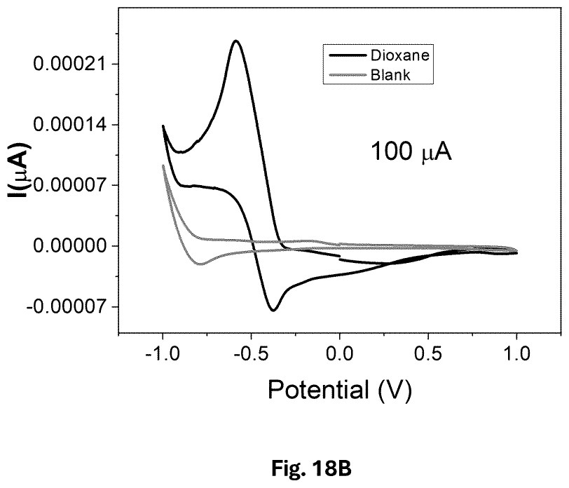

Plausible pathway for quenching by photoinduced electron transfer (PET) due to dioxane coordination to Eu³⁺ ion, has been realized by developing energy level diagram of the involved species. The diagram is shown in FIG. 16. The valence band positions of MSBCN and MZN-2 were obtained from Valence band XPS (FIG. 17), the energy band gap were calculated from respective τauc's plot (FIG. 18A) and conduction band positions were calculated by using Eq. 1. Whereas for the analyte dioxane, cyclic voltammetry (FIG. 18B) was carried out to find out the oxidation and reduction potential and the following equation (Eq. 2) was used to calculate HOMO and LUMO energy levels. Standard data was available for the other ligand BDC. In the MOF composition, g-C₃N₄ is present with highly conjugated 3-D network system. Also, presence of carboxyl functional group with enriched electron density in BCN provides a pool of delocalized electron density to the MOF composition. This constitutes the HOMO of the MOF sensor while vacant d-orbitals of Eu³⁺ ion contribute to the LUMO of the same. From the energy level diagram in FIG. 16, it is observed to be Type I band arrangement after dioxane addition, where the electrons from conduction band of MZN-2 can easily transfer to the conduction band of dioxane causing quenching in fluorescence due to relaxation of singly excited electrons from conduction band to valence band.

$${E}_{CB} = {E}_{VB} - {E}_{g} \qquad ( 1 )$$

$${E}_{HOMO} = - e [ {E}_{ox}^{onset} + 4.4 ] \qquad ( 2 )$$

$${E}_{LUMO} = - e [ {E}_{red}^{onset} + 4.4 ]$$

Hence formation of ground state complex (GSC) results in more effective PET causing efficient quenching of metal centered emissions. Whereas on GSC formation between dioxane and MZN-2, the electronic transition viz. n to π* increases which ultimately results in enhancement of absorbance in the UV-visible spectra and ligand centered emission at 504 nm. As a consequence of all these phenomena, a ratiometric sensing behavior is exhibited by the MOF MZN-2 towards 1,4-dioxane.

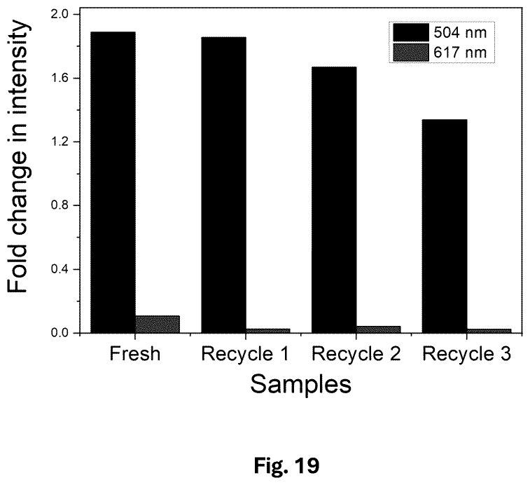

The recyclability of the MZN-2 MOF sample was established for ratiometric fluorescence sensing of dioxane up to fourth cycle without significant loss in activity. The results are presented in FIG. 19.

### Example 3B

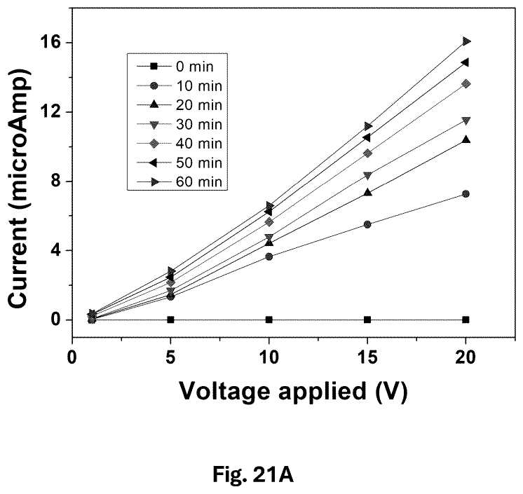

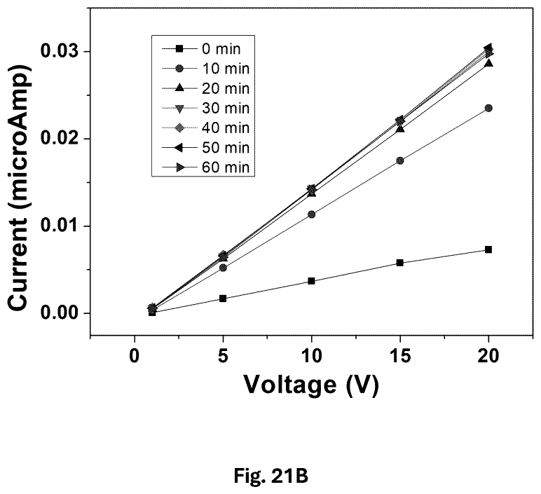

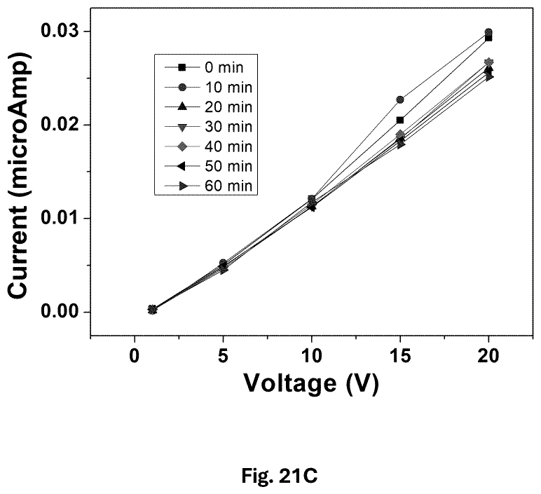

Then we explore the potential of the developed MOF whether they can behave as a direct electrical sensor for the industrial gases. The sensor response was recorded in terms of the electrical current signal change in the gaseous environment at room temperature. For that the powder MOF sample was compressed to a pellet of thickness 1.2±2 mm and diameter of 1.3 cm. The gas chamber system was constructed on a vacuum desiccator of DN 140 mm by modifying the top portion so that the custom-built 2-point probe can be connected. The sample pellet was loaded into this 2-point probe system and the electrical response was real-time recorded with the help of an electrometer (2401, Keithley). The outline of the experimental setup was drawn in FIG. 20. At first, the electrical signal of the pellet was recorded against increasing voltage input in air. We allowed 30 sec to stabilize the signal before recording. Then sensing gas environment of dioxane and methanol were prepared by keeping a petri dish filled with respective liquids at the base of the desiccator. And after loading the sample pellet in this environment air inside the desiccator was evacuated with the help of a vacuum pump. The results in the form of I-V characteristic curves are shown in FIG. 21A-21C, from where excellent sensing response is clearly observed for both methanol (FIG. 21A) and dioxane (FIG. 21B). But in air, the signal remains almost constant in the same setup (FIG. 21C). The developed MOF possesses high oxygen functionalities with hydrophilicity on its surface. Therefore, adsorption of volatile organic compounds is induced by hydrogen bonding interactions. Since methanol is a highly polar solvent so its adsorption is more on MOF surface leading to higher response in electrical signal.

### ADVANTAGES

The main advantage of this invention are:
a) The role of structure-directing agent can be played by functionalized g-C₃N₄ in MOF formation without using any organic molecule.
b) The fluorescence sensing exhibited by the MOF towards environmental toxin 1,4-dioxane is selectively ratiometric.
c) The sensing activity can be monitored through color change under UV light, which does not require any sophisticated equipment.
d) Sensing of an environmental toxin both in liquid and gaseous phase by using the same material has the advantage of complete control over the sensing ability of target compound.
e) Presence of semiconducting g-C₃N₄ phase in the developed MOF renders an additional property of photocatalysis which can be explored.

What is claimed is:

1. A fluorescent material comprising:
a europium precursor; and
a structure directing ligand comprising functionalized graphitic carbon nitride (g-C₃N₄).

2. The fluorescent material as claimed in claim 1, further comprising transition metal disulfides (TMD).

3. The fluorescent material as claimed in claim 2, further comprising an organic molecule.

4. The fluorescent material as claimed in claim 1, wherein the fluorescent material has an average particle size in a range of 50 nm to 200 nm.

5. The fluorescent material as claimed in claim 2, wherein the TMD is molybdenum disulfide (MoS₂) and the organic molecule is terephthalic acid or 2-aminoterephthalic acid.

6. The fluorescent material as claimed in claim 1, wherein the fluorescent material is characterized to exhibit dual emission in cyan region and orange-red region upon excitation by UV light.

7. A process for making a fluorescent material, the process comprising:
functionalizing graphitic carbon nitride (g-C₃N₄) with benzoic acid to form functionalized g-C₃N₄ (BCN);
reacting BCN with molybdenum disulfide (MoS₂) to make a composite (MS-BCN);
dispersing a europium precursor and the composite MS-BCN in a solvent to form a mixture;
adding an organic acid to the mixture, and stirring at room temperature for about an hour;
autoclaving at 130° C. for about 48 hours, and centrifuging to form a precipitate;
washing the precipitate with ethanol-acetone; and
oven drying the precipitate at 60° C. for 12 hours to form the fluorescent material (MZN-2) as a cream-coloured powder.

8. The process as claimed in claim 7, wherein the europium precursor is selected from the group consisting of europium (III) chloride hexahydrate, europium (III) chloride, europium (III) acetate, europium (III) carbonate, europium (III) nitrate, europium (III) oxalate, europium (III) carbonate, europium (III) oxide and, a combination thereof.

9. The process as claimed in claim 7, wherein the solvent is N,N-dimethylformamide (DMF), N,N-diethyl-formamide (DEF), or 1-methyl-2-pyrrolidone (NMP).

10. The process as claimed in claim 7, wherein the BCN and MoS₂ are in a weight ratio in a range of about 1% (w/w) to 7% (w/w).

11. The process as claimed in claim 7, wherein the MS-BCN and the europium precursor are in a weight ratio in a range of about 1% (w/w) to 7% (w/w).

12. The process as claimed in claim 7, wherein the europium precursor and the organic acid are in mole ratio of 3:1.

13. The process as claimed in claim 7, wherein the organic acid is terephthalic acid or 2-aminoterephthalic acid.

14. A process for using a fluorescent material to sense volatile organic compounds (VOC) in a sample, the process comprising:
preparing the fluorescent material, wherein the fluorescent material comprises a europium precursor, and a structure directing ligand comprising functionalized graphitic carbon nitride (g-C₃N₄); and
sensing the VOC in the sample using the fluorescent material.

15. The process as claimed in claim 14, wherein sensing the VOC comprises ratiometric sensing and a gaseous phase sensing separately or simultaneously.

16. The process as claimed in claim 14, wherein the VOC is 1,4-dioxane.

17. The process as claimed in claim 16, wherein the ratiometric sensing of 1,4-dioxane comprises:
preparing a solution of the fluorescent material in water;
adding a sample comprising 1,4-dioxane to the solution;
recording fluorescence spectra of the solution by exciting with UV light; and
detecting colorimetric change of the solution from pink to blue under UV light before and after adding the sample.

18. The process as claimed in claim 17, wherein limit of detection (LOD) of 1,4-dioxane is about 0.3 μM, and wherein the fluorescent material is capable of selectively detecting 1,4-dioxane from a pool of solvents comprising water, ethanol, methanol, acetonitrile, ethyl acetate, N,N-dimethylformamide, propanol, isopropanol, butanol, dichloromethane and a combination thereof.

19. The process as claimed in claim 17, wherein the fluorescent material is re-used at least up to 4 times.

20. The process as claimed in claim 16, wherein the gaseous phase detection of 1,4-dioxane comprises monitoring changes in current vs. voltage (I-V) characteristics of the fluorescent material in air and in the presence of 1,4-dioxane.
